(19) United States
(12) Patent Application Publication
(10) Pub. No.: US 2026/0104411 A1
(43) Pub. Date: Apr. 16, 2026

(54) HIGH-AFFINITY NUCLEIC ACID APTAMER FOR HISTAMINE AND AQUATIC PRODUCT FRESHNESS ENZYME SENSOR

(71) Applicant: OCEAN UNIVERSITY OF CHINA, Qingdao City (CN)

(72) Inventors: Xiangzhao MAO, Qingdao City (CN); Sai WANG, Qingdao City (CN); Kaiyue WEI, Qingdao City (CN); Wenjing WANG, Qingdao City (CN); Rui FENG, Qingdao City (CN)

(21) Appl. No.: 19/282,113

(22) Filed: Jul. 28, 2025

(30) Foreign Application Priority Data
Jul. 30, 2024 (CN) 202411025191.7

(51) Int. Cl. G01N 33/543; C12N 15/115; G01N 21/78; G01N 33/12; G01N 33/53

(52) U.S. Cl. CPC G01N 33/54346; C12N 15/115; G01N 21/78; G01N 33/12; G01N 33/5308; C12N 2310/16

(57) ABSTRACT

A high-affinity nucleic acid aptamer for histamine and an aquatic product freshness enzyme sensor, belonging to the technical field of biological detection, is named HIS3-T2 and has a nucleotide sequence shown in SEQ ID NO.2. An application of the high-affinity nucleic acid aptamer for histamine in detecting, separating or enriching the histamine. The aquatic product freshness enzyme sensor includes the following components: an aptamer HIS3-T2, chitopentaose, a nanozyme AuNPs@FeP, and a TMB chromogenic solution. Applications of the aquatic product freshness enzyme sensor in detecting the histamine and in rapidly evaluating freshness of an aquatic product. The present invention further provides a method for rapidly evaluating freshness of an aquatic product. The present invention provides a new biological recognition element for detection of the histamine, which has great significance for the detection and enrichment of the histamine.

### REFERENCE TO AN ELECTRONIC SEQUENCE LISTING

The content of the electronic sequence listing (Sequence List-GQ-US-2025-01; Size: 2,747 bytes; and Date of Creation: Jul. 18, 2025) is herein incorporated by reference in its entirety.

### TECHNICAL FIELD

The present invention relates to a high-affinity nucleic acid aptamer for histamine and an aquatic product freshness enzyme sensor, belonging to the technical field of biological detection.

### BACKGROUND

Histamine is a biogenic amine primarily generated through oxidative decarboxylation of L-histidine. When aquatic products are improperly handled or stored, they typically exhibit high concentrations of histamine. Excessive intake of such foods may increase the accumulation of histamine in the human body, leading to foodborne diseases and triggering allergic reactions such as diarrhea, hypertension, headache, nausea, and vomiting. Therefore, histamine can be used as a detection indicator for deterioration of the aquatic products.

Nucleic acid aptamers are a class of single-stranded nucleotides with specific recognition capabilities, which can achieve high-specificity and high-affinity binding to targets. HIS3, a nucleic acid aptamer for histamine has insufficient affinity, thereby requiring modification to enhance the affinity thereof. Truncation-based aptamer optimization strategies have been proven to effectively enhance the affinity for detection of small molecular targets. However, truncation usually requires multiple attempts to obtain the desired high-affinity aptamer, and simpler and more reasonable optimization strategies remain to be explored.

Nucleic acid aptamer-based detection is usually combined with various transduction methods such as colorimetry, fluorescence methods, and electrochemical techniques. Enzyme sensors based on the nucleic acid aptamers and nanozymes are one of the current research hotspots.

### SUMMARY

In response to the prior art mentioned above, the present invention provides a high-affinity nucleic acid aptamer for histamine and an aquatic product freshness enzyme sensor, belonging to the technical field of biological detection.

The present invention is achieved by the following technical solution:

A high-affinity nucleic acid aptamer for histamine, where the high-affinity nucleic acid aptamer is named HIS3-T2 and has a nucleotide sequence shown in SEQ ID NO. 2.

An application of the high-affinity nucleic acid aptamer for histamine in detecting, separating, or enriching the histamine, or in preparing a product for detecting, separating or enriching the histamine.

Further, the product may be in the form of a reagent, a kit or a sensor.

An aquatic product freshness enzyme sensor, including the following components: an aptamer HIS3-T2, chitopentaose, nanozyme AuNPs@FeP, and a TMB chromogenic solution, wherein the nucleotide sequence of the aptamer HIS3-T2 is shown in SEQ ID NO.2.

Further, the enzyme sensor consists of the following components: 10 μL of an aptamer HIS3-T2 solution at a concentration of 1 μM, 10 μL of a chitopentaose solution at a concentration of 0.33 μM, 90 μL of a nanozyme AuNPs@FeP solution at a concentration of 1.98 nM, and 50 μL of the TMB chromogenic solution, which are amounts required for a single sample detection.

Further, the TMB chromogenic solution is prepared from TMB (3,3′,5,5′-tetramethyl benzidine), H₂O₂, and an acetic acid buffer, where in the chromogenic solution, the TMB has a concentration of 2 mM, the H₂O₂ has a volume concentration of 20%, and the acetic acid buffer has a concentration of 1 M and pH of 4.0.

Further, the nanozyme AuNPs@FeP solution is prepared by the following method:
(1) preparation of gold nanoparticles: adding 1 mL of a chloroauric acid solution at a concentration of 10 mg/mL to 95 mL of ultrapure water, heating to boiling, adding 4 mL of a trisodium citrate solution at a concentration of 10 mg/mL, and continuing heating until the solution turns a stable wine-red color to obtain an AuNPs solution; and
(2) preparation of the nanozyme AuNPs@FeP solution: taking 496 μL of the AuNPs solution, adding 2 μL of a phosphate buffer at a concentration of 1 mM and with pH of 7.1, stirring at room temperature for 3 min, then adding 2 μL of an FeCl₂ solution at a concentration of 50 μM, and reacting for 15 min to obtain the nanozyme AuNPs@FeP solution, where AuNPs@FeP has a concentration of 1.98 nM.

An application of the aquatic product freshness enzyme sensor in detecting the histamine.

An application of the aquatic product freshness enzyme sensor in rapidly evaluating freshness of an aquatic product.

A method for rapidly evaluating freshness of an aquatic product, including: using microneedle patches to perform pressing extraction on an aquatic product to be detected for 2 min, then transferring the moist microneedle patches to a centrifuge tube containing 995 μL of a PBS buffer at a concentration of 1 mM, centrifuging at 4500 rpm for 10 s, and filtering to obtain a sample to be detected; and detecting a concentration of histamine in the sample to be detected by using the aquatic product freshness enzyme sensor.

Further, the microneedle patches are prepared by the following method: taking 13 g of a polyvinyl alcohol prepolymer same in 100 mL of ultrapure water at 90° C., cooling to 60° C., and adding 0.5 g of hyaluronic acid; and then, pouring a mixed solution into a mold for forming a cavity array of microneedles, defoaming for 10 min under a condition of −1.0 Mpa, and drying to obtain the microneedle patches.

According to the present invention, by modifying an original aptamer HIS3, an aptamer HIS3-T2 with higher affinity for histamine is obtained. The aptamer HIS3-T2 has a K_{d} value of 59.48 nM, representing an affinity increase of 25.97 times compared to the original aptamer HIS3. The aquatic product freshness enzyme sensor of the present invention can specifically detect histamine and can be used for rapidly evaluating the freshness of the aquatic product. The present invention provides a new biological recognition element for detection of the histamine, which has great significance for the detection and enrichment of the histamine.

Various terms and phrases used in the present invention have general meanings known to those skilled in the art.

### BRIEF DESCRIPTION OF THE DRAWINGS

FIG. 1: a schematic diagram of affinity detection results between an original aptamer HIS3 and histamine.

FIG. 2: a schematic diagram of a secondary structure of the original aptamer HIS3.

FIG. 3: a schematic diagram of molecular docking results between an original aptamer HIS3 and histamine.

FIG. 4: a schematic diagram of a secondary structure of an aptamer HIS3-T2.

FIG. 5: a schematic diagram of affinity detection results between an aptamer HIS3-T2 and histamine.

FIG. 6: a standard curve of histamine concentration-signal suppression rate.

FIG. 7: a schematic diagram comparing the signal suppression rates of histamine, spermidine (Spm), cadaverine (Cad), putrescine (Put), and 5-hydroxytryptamine (5′-HT).

FIG. 8: a schematic diagram comparing the detected histamine concentrations and actual histamine concentrations.

FIG. 9: a schematic diagram of comparison between detection results of salmon samples.

FIG. 10: a schematic diagram of comparison between detection results of shrimp samples.

FIG. 11: swelling rates of the microneedle patches.

FIG. 12: a schematic diagram of weight determination results of the microneedle patches.

FIG. 13: a schematic diagram of tolerance determination results of the microneedle patches.

### DETAILED DESCRIPTION OF THE EMBODIMENTS

The present invention is further described below in conjunction with the embodiments. However, the scope of the present invention is not limited to the following embodiments. Those skilled in the art will recognize that various changes and modifications can be made to the present invention without departing from the scope of the present invention.

Unless otherwise specified, all instruments, reagents, materials, experimental methods, and detection methods described in the following embodiments are conventional and commercially available, or are known in the prior art.

Biolayer interferometry (BLI) molecular interaction analyzer is a widely used tool for quantifying the affinity of nucleic acid aptamers for their targets. This instrument quantitatively analyzes molecular interaction strength based on BLI principles, a key technique in contemporary biomolecular interaction research.

The nucleic acid aptamer is immobilized on a surface of a sensor through the binding of biotin and streptavidin. A buffered solution for reaction, a biotin-labeled nucleic acid aptamer, and different concentrations of targets are added to a 96-well plate, and the instrument is set up with the following procedure: sensor equilibrium for 120 s, nucleic acid aptamer immobilization for 600 s, sensor equilibrium for 120 s, target binding for 120 s, target dissociation for 120 s, a temperature being 25° C., and a frequency being 2 Hz. An affinity constant (Kd value) can be obtained by fitting obtained binding-dissociation curves.

### Embodiment 1 Modification of a Nucleic Acid Aptamer for Histamine

An original aptamer HIS3 for histamine had a nucleotide sequence shown in SEQ ID NO. 1 as follows (orientation 5′-3′):

TGAGCCCAAGCCCTGGTATGAAAGTCGCGGTAAGGAGCGTGCTTGG ATTCTCGTTGGGCTGGCAGGTCTACTTTGGGATC, which are 80 bases in total.

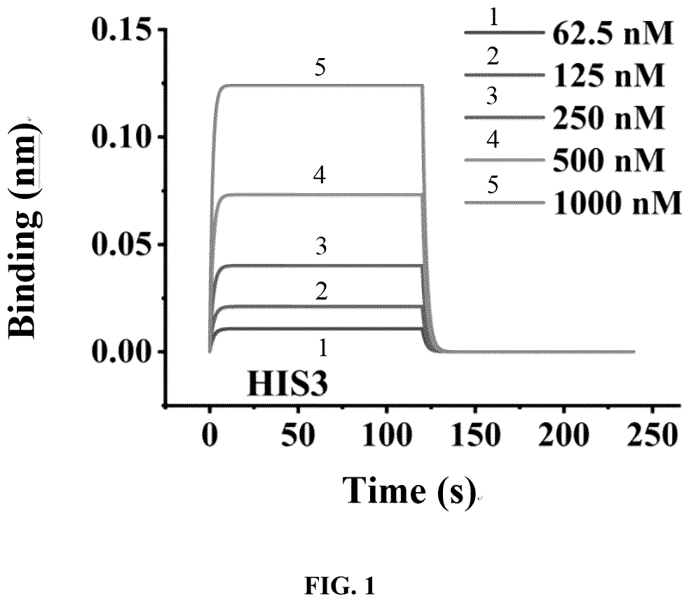

An affinity of the original aptamer HIS3 for histamine was determined through BLI experiments, and detection results were shown in FIG. 1, with a Kd value of 2.29 μM. The Kd value of the original aptamer HIS3 was not ideal. To obtain a nucleic acid aptamer with better affinity, the present invention attempted to modify the original aptamer.

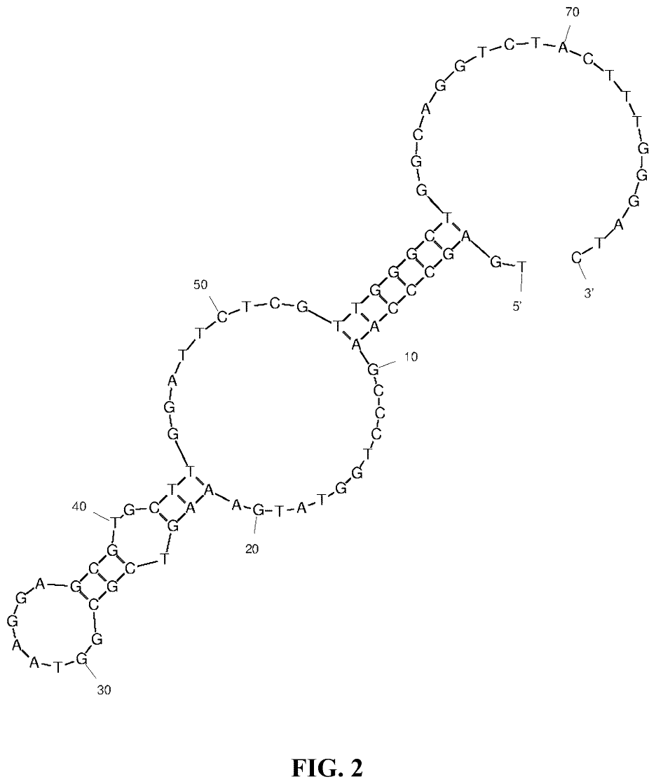

A secondary structure of the original aptamer HIS3 was predicted by using an online analysis tool Mfold web server, and a sequence in pdb format was downloaded from RNAComposer. A schematic diagram of the secondary structure of the original aptamer HIS3 was shown in FIG. 2. Through analysis, it was found that a middle part of a stem loop of the original aptamer HIS3 contained a terminal-locking structure.

A binding mechanism between the original aptamer HIS3 and histamine was analyzed through molecular docking. Prior to docking, a histamine molecular file in MOL2 format was prepared. A lattice energy calculation file was established. A docking calculation region was set to encompass all molecules, and calculation lattice points were set with a spacing of 1 Å. Molecular interactions between the aptamer and histamine were explored based on a Lamarkian genetic algorithm and starting from the minimum binding free energy, and were subjected to visualized analysis by using PyMOL.

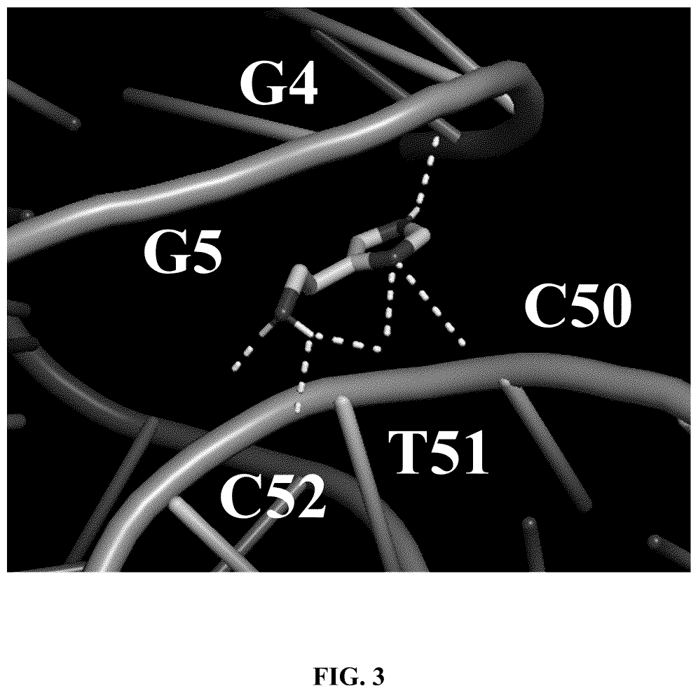

A schematic diagram of molecular docking results between the original aptamer HIS3 and the histamine was shown in FIG. 3. Key binding sites G4, G5, C50, T51, and C52 of the original aptamer HIS3 and the histamine were located in a terminal-locking structure region in the middle part of the stem loop. The terminal-locking structure was directly separated to obtain a nucleic acid aptamer named aptamer HIS3-T2. The aptamer HIS3-T2 had a nucleotide sequence shown in SEQ ID NO. 2 as follows (orientation 5′-3′):

TGAGCCCAAGCCCTGGTATGAATGGATTCTCGTTGGGCTGG, which are 41 bases in total.

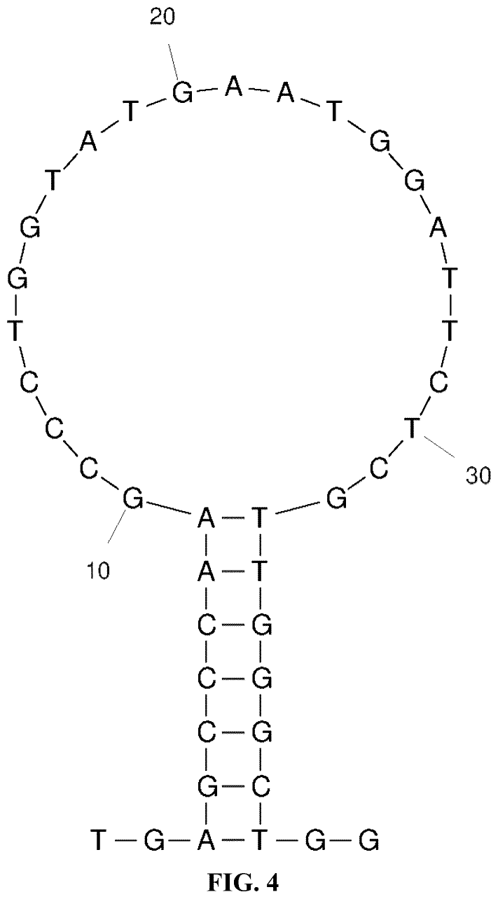

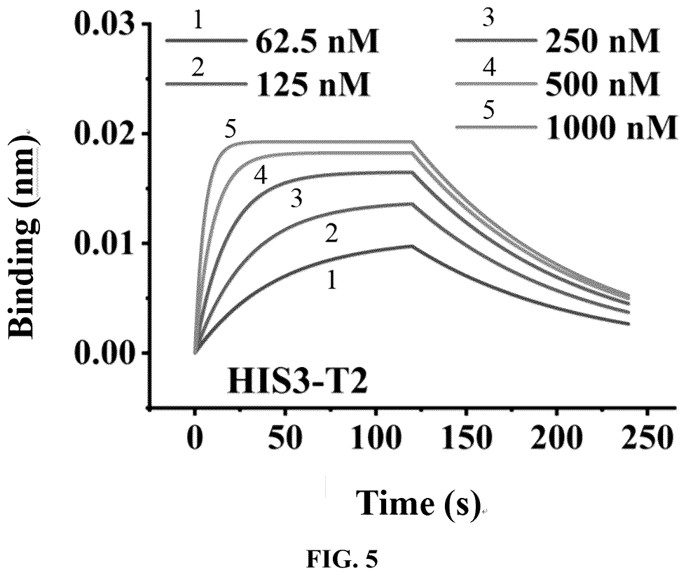

A schematic diagram of a secondary structure of the aptamer HIS3-T2 was shown in FIG. 4. An affinity of the aptamer HIS3-T2 for histamine was determined through BLI experiments, and detection results were shown in FIG. 5, with a Kd value of 59.48 nM. Compared to the original aptamer HIS3, the aptamer HIS3-T2 had an affinity increased by 25.97 times.

The above results indicated that for nucleic acid aptamers with multiple stem loops, a terminal-fixed structure separation strategy was adopted to extract a specific structure of the original aptamer HIS3, yielding the aptamer HIS3-T2 with higher affinity. According to the strategy adopted in the present invention, after performing molecular docking analysis on binding sites between the aptamer and a target, the terminal-locking structure was directly extracted under the principle of not destroying the binding sites, so as to obtain a high-affinity aptamer. Unlike conventional truncation methods requiring iterative optimization, the strategy herein provides a rational, single-step solution that significantly reduces experimental workload and costs.

### Embodiment 2 Construction of an Aquatic Product Freshness Enzyme Sensor

An aquatic product freshness enzyme sensor consisted of the following solutions: 10 μL of an aptamer HIS3-T2 solution at a concentration of 1 μM, 10 μL of a chitopentaose solution at a concentration of 0.33 μM, 90 μL of an AuNPs@FeP solution at a concentration of 1.98 nM, and 50 μL of a TMB chromogenic solution, which were amounts required for a single sample detection.

The TMB chromogenic solution was prepared from TMB, H₂O₂, and an acetic acid buffer (pH 4.0, 1 M), where in the chromogenic solution, the TMB had a concentration of 2 mM, and the H₂O₂ had a volume concentration of 20%.

A preparation method for the AuNPs@FeP solution was as follows:
(1) preparation of gold nanoparticles: adding 1 mL of a chloroauric acid solution at a concentration of 10 mg/mL to 95 mL of ultrapure water, heating to boiling, rapidly adding 4 mL of a trisodium citrate solution at a concentration of 10 mg/mL, and continuing heating until the solution turned a stable wine-red color to obtain an AuNPs solution; and
(2) preparation of the AuNPs@FeP solution: taking 496 μL of the AuNPs solution, adding 2 μL of a phosphate buffer at a concentration of 1 mM and with pH of 7.1, stirring at room temperature for 3 min, then adding 2 μL of an FeCl₂ solution at a concentration of 50 μM, and reacting for 15 min to obtain the AuNPs@FeP solution, where AuNPs@FcP had a concentration of 1.98 nM.

An application method for the aquatic product freshness enzyme sensor constructed by the present invention was as follows:
(1) mixing a histamine reference standard solution with HIS3-T2 and reacting for 30 min; adding a chitopentaose solution and reacting for 10 min; adding an AuNPs@FeP solution and incubating in the dark for 5 min to obtain a mixture; taking 50 μL of the mixture, adding 50 μL of a TMB chromogenic solution, and measuring an absorbance value at 650 nm by using a microplate reader; and detecting the absorbance values of the histamine reference standard solutions at different concentrations, calculating signal suppression rates (A0−Ai)/A0(%), and drawing a standard curve.

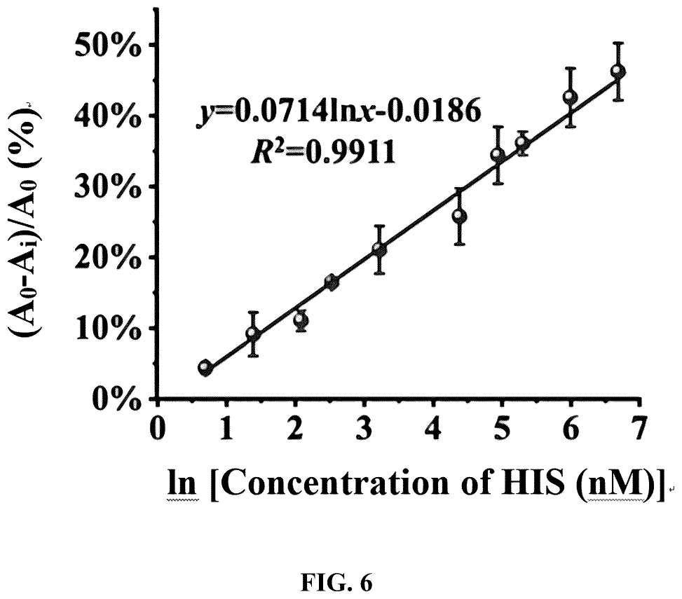

A concentration range of the histamine reference standard solution was 2 nM to 800 nM, and the signal suppression rate ΔA=(A0−Ai)/A0(%), where A0 represented an absorbance value of blank (a histamine concentration being 0), and Ai represented the absorbance values of the histamine reference standard solutions at different concentrations. The standard curve of histamine concentration-signal suppression rate was shown in FIG. 6, and a corresponding regression equation was y=0.0714lnx−0.0186, R²=0.9911, where y was a signal suppression rate at 650 nm, x was the histamine concentration, and a detection limit was 1.89 nM.

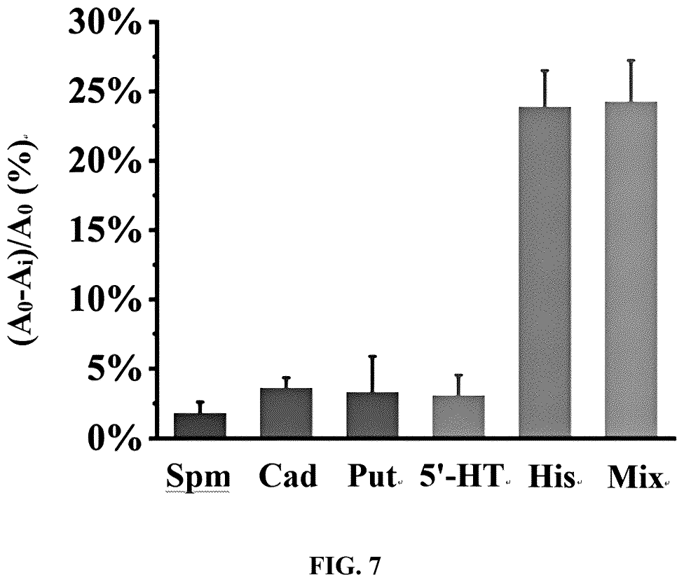

An 80 nM histamine reference standard solution as well as 80 nM reference standard solutions of the other four biogenic amines (spermidine, cadaverine, putrescine, and 5-hydroxytryptamine) were detected, the absorbance value at 650 nm was measured by using a microplate reader, and the signal suppression rate ΔA (as above) was calculated. A schematic diagram of comparison among the signal suppression rates of histamine, spermidine, cadaverine, putrescine, and 5-hydroxytryptamine was shown in FIG. 7, and only histamine showed obvious signal suppression, indicating that the aquatic product freshness enzyme sensor constructed by the present invention has excellent specificity.

(2) mixing a sample to be detected with HIS3-T2 and reacting for 30 min; adding the chitopentaose solution and reacting for 10; adding the AuNPs@FeP solution and incubating in the dark for 5 min to obtain a mixture; taking 50 μL of the mixture, adding 50 μL of the TMB chromogenic solution, measuring the absorbance value at 650 nm by using a microplate reader, calculating a signal suppression rate, substituting same into the standard curve, and calculating a concentration of the histamine in the sample to be detected.

The aquatic product freshness enzyme sensor constructed by the present invention had the following detection principle: when a reaction system did not contain histamine, chitopentaose bound to the aptamer HIS3-T2, only a slight excess of chitopentaose was adsorbed on AuNPs@FeP to form AuNPs@FeP-COS, and due to its high enzymatic activity, the AuNPs@FeP-COS catalyzes the TMB chromogenic solution to produce darker colors. When the reaction system contained histamine, an aptamer-target recognition behavior was triggered, leading to conformational changes in a tertiary structure of the aptamer, which was more favorable for binding to chitopentaose. The activity of AuNPs@FeP-COS was positively correlated with the concentration of histamine, resulting in a significant color change of a substrate. As the concentration of histamine increased, the formation of AuNPs@FeP-COS decreased and the color after a catalytic reaction kept getting lighter. Preliminary quantitative analysis of histamine was achieved under optimal experimental conditions.

### Embodiment 3 Practical Application of the Aquatic Product Freshness Enzyme Sensor

Samples to be detected were extracted by using microneedle patches, and the histamine was detected by using the aquatic product freshness enzyme sensor constructed in Embodiment 2, as specifically described below.

(1) prior to actual sample detection, 1% agarose hydrogels containing different concentrations of histamine (8 nM, 41 nM, 80 nM, 146 nM, and 208 nM) were used for feasibility verification, and the microneedle patches were used to perform pressing extraction thereon for 2 min, respectively, and then the moist microneedle patches were transferred to a centrifuge tube containing 995 μL of a PBS buffer at a concentration of 1 mM, centrifugation was performed at 4500 rpm for 10 s, and filtration was performed to obtain the samples to be detected.

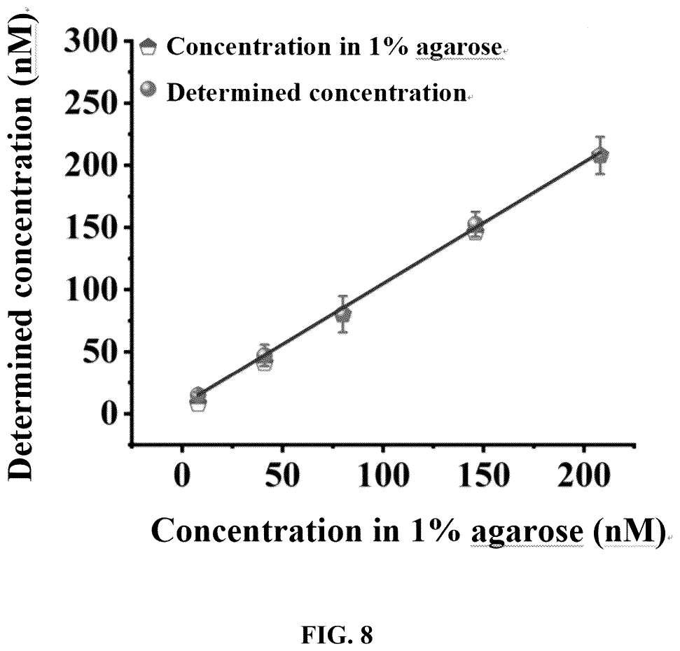

The concentrations of histamine in the samples to be detected were detected with the aquatic product freshness enzyme sensor, and a linear correlation equation of y=0.9752x+6.3812 (R²=0.9986) was derived, where x represented the concentrations (nM) of histamine in the 1% agarose hydrogels and y represented the concentrations (nM) of histamine detected with the aquatic product freshness enzyme sensor. A schematic diagram of comparison between the detected histamine concentrations and actual histamine concentrations was shown in FIG. 8, and the detected histamine concentrations had good consistency with the actual histamine concentrations.

The extraction time was 2 min, and a polyvinyl alcohol concentration of the microneedle patches was 13%. A preparation method for the microneedle patches was detailed in Embodiment 4 below.

(2) Practical application:

Salmon samples (back tissue and muscle) and shrimp samples (muscle tissue near the head) were placed in disposable petri dishes and sealed, respectively. The petri dishes were stored at 20° C. for 5 days, and samples were taken once every 12 h. The tissues were punctured and then subjected to pressing extraction for 2 min by using the microneedle patches, and then the moist microneedle patches were transferred to a centrifuge tube containing 995 μL of a PBS buffer at a concentration of 1 mM, centrifugation was performed at 4500 rpm for 10 s, and filtration was performed to obtain the samples to be detected.

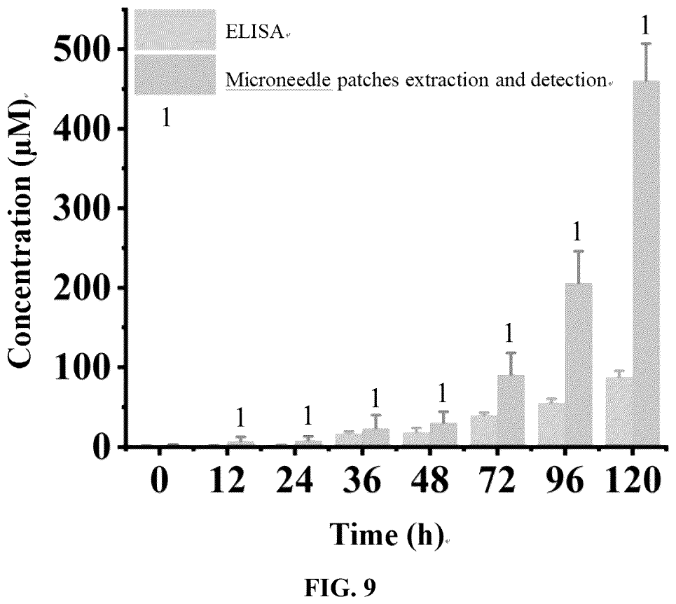

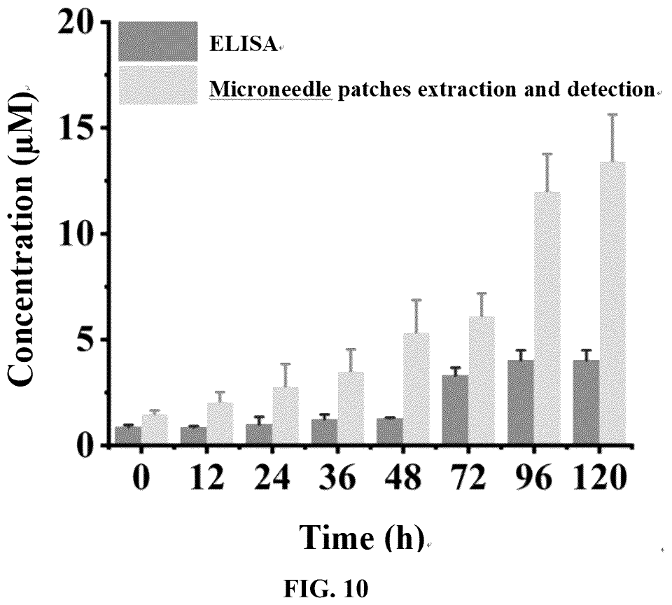

The concentrations of histamine in the samples to be detected were detected with the aquatic product freshness enzyme sensor, and detection results were compared to those obtained from a commercial ELISA detection kit (routinely purchased, a histamine (HIS) enzyme-linked immunosorbent assay kit, Manufacturer: BBI Life Sciences Corporation, Product Code: D751012, Packing Size: 96 TESTS), for evaluating the accuracy of extraction performed by using the microneedle patches. A schematic diagram of comparison between the detection results of the salmon samples was shown in FIG. 9, and a schematic diagram of comparison between the detection results of the shrimp samples was shown in FIG. 10. It could be seen that higher concentrations of histamine could be extracted by the microneedle patches, which might be due to a better extraction ability of the microneedle patches. During an initial storage period of salmon and shrimp samples, the content of histamine was relatively low. With extension of storage time, the concentration of histamine showed a significant increase trend. At the end of storage, the content of histamine in salmons increased significantly by 190 times, while detection results of collected shrimp samples only showed an increase of about 9 times. The increase in the histamine content observed during storage of the salmons might be attributed to their higher protein content. Moreover, certain bacteria present in salmon bodies in this situation might produce enzymes that promoted the conversion of histidine to histamine. Therefore, as the deterioration degree of the salmons increased, the number of the bacteria also increased, thus further promoting the conversion of histidine to histamine.

### Embodiment 4 Preparation and Optimization of the Microneedle Patches

A certain amount of a polyvinyl alcohol prepolymer (11 g, 12 g, 13 g, 14 g, 15 g) was taken, dissolved in 100 mL of ultrapure water at 90° C., and cooled to 60° C., and 0.5 g of hyaluronic acid was added; and then, a mixed solution was poured into a mold (the mold being made of polydimethylsiloxane) for forming a cavity array of microneedle patches, and defoaming was performed for 10 min under a condition of −1.0 Mpa. Subsequently, the prepared microneedle patches were dried at 37° C. for 5 h to form intramolecular and intermolecular hydrogen bonds. A filling-drying step was repeated once, and drying was performed continuously for 12 h to obtain microneedle patches with different polyvinyl alcohol concentrations (11%, 12%, 13%, 14%, 15%, in g/mL). The microneedle patches were carefully removed from the mold, and were sealed and stored in a dry condition.

For the prepared microneedle patches, a nanoneedle was conical in shape and had a height of 600 μm and a bottom diameter of 333 μm. A distance between center lines of two needles was approximately 386 μm.

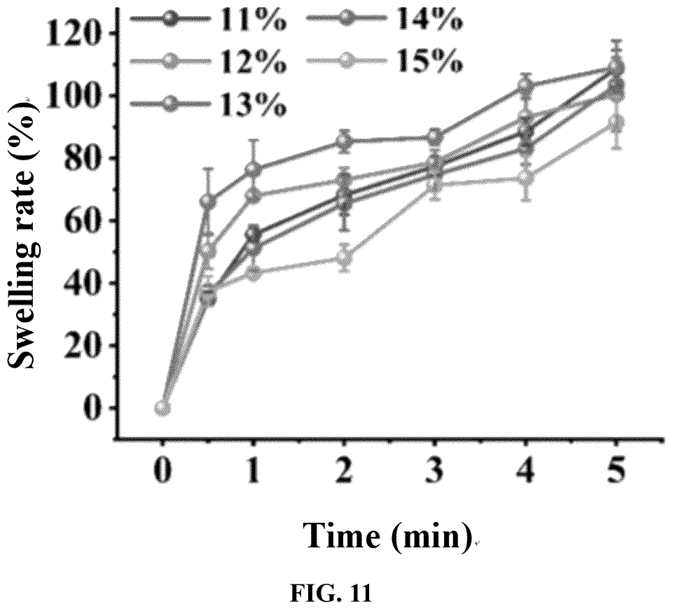

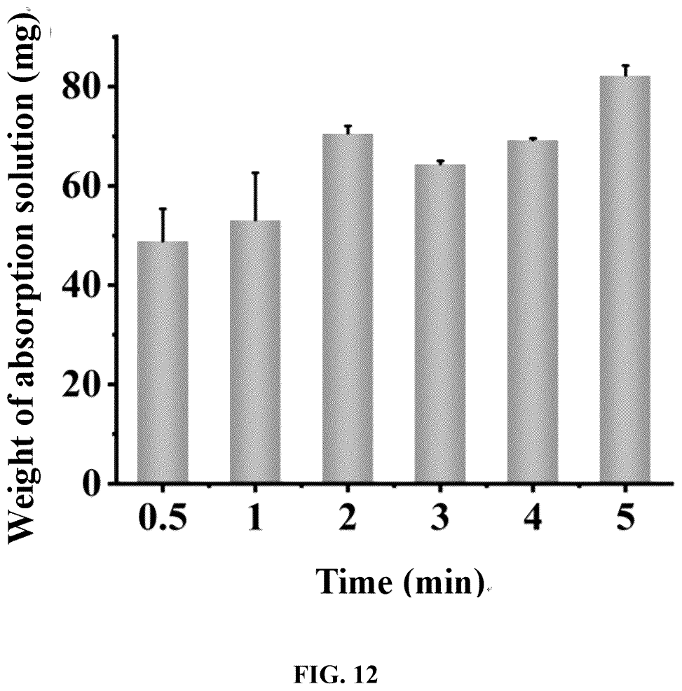

The 1% agarose hydrogel was taken as an extraction object, and the microneedle patches were used to perform pressing extraction for 1-5 min for sampling. The effects of the polyvinyl alcohol concentration and extraction time on the swelling ability of the microneedle patches were explored. The swelling rates of the microneedle patches were shown in FIG. 11. It could be seen that the swelling ability showed an increasing trend when the polyvinyl alcohol concentration was within a range of 11% to 13%. As the polyvinyl alcohol concentration increased, the swelling performance decreased, which was related to the high degree of cross-linking. In addition, since a three-dimensional network structure inside the microneedle patches was destructed, and intramolecular and intermolecular hydrogen bonds were broken, tips of the microneedle patches dissolved. Therefore, an extraction time of 2 min was determined to be the optimal duration. The weight after absorption of a histamine solution was determined with the microneedle patches having a polyvinyl alcohol concentration of 13%, and a schematic diagram of weight determination results of the microneedle patches was shown in FIG. 12. After swelling, the microneedle patches exhibited equilibrium absorption of more than 70 mg of liquid.

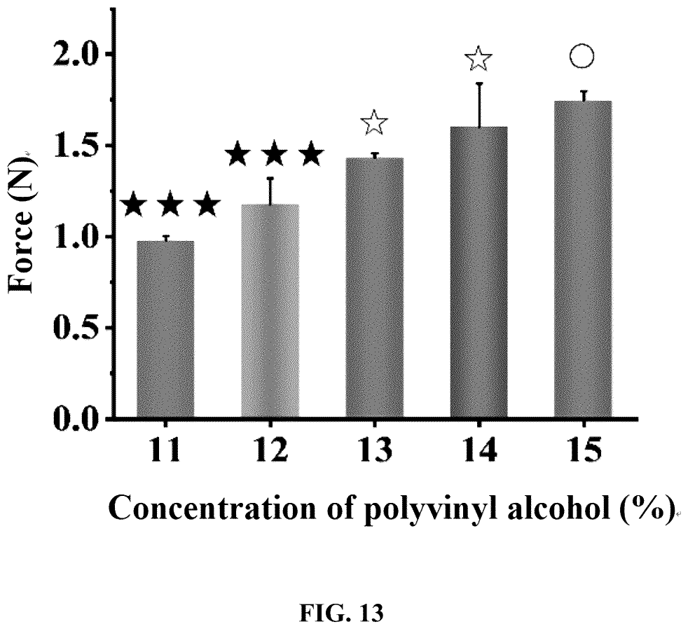

A texture analyzer (TMS-Touch, FTC, USA) was used to study the mechanical properties of the microneedle patches. A probe selected was TMS12.7 mm Perplex. Parameter setting: a measurement mode and option were gel; a triggering force was 0.2 N; a speed at which a force biosensor approached and left the sample was 60 mm/min; a measurement rate was 60 mm/min; and when the biosensor moved 0.5 mm, the measurement ended. The tolerance of cross-linked microneedle patches was studied by compression experiments, and tolerance determination results of the microneedle patches were shown in FIG. 13. It could be seen that as the polyvinyl alcohol concentration increased, the tolerance was enhanced, and the tolerance was 1.43±0.03 N/needle when the polyvinyl alcohol concentration was 13%.

The microneedle patches with a polyvinyl alcohol concentration of 13% were selected for the experiments by comprehensively considering the determination results of the swelling ability and mechanical strength.

The foregoing embodiments are provided for those skilled in the art to fully disclose and describe how the claimed embodiments are implemented and used, rather than to limit the scope of the disclosure herein. Modifications that would be obvious to those skilled in the art shall fall within the scope of the appended claims.

1. A high-affinity nucleic acid aptamer for histamine, wherein the high-affinity nucleic acid aptamer is named HIS3-T2 and has a nucleotide sequence shown in SEQ ID NO.2.

2. An application of the high-affinity nucleic acid aptamer for histamine according to claim 1 in detecting, separating, or enriching the histamine, or in preparing a product for detecting, separating or enriching the histamine.

3. An aquatic product freshness enzyme sensor, comprising the following components: an aptamer HIS3-T2, chitopentaose, a nanozyme AuNPs@FeP, and a TMB chromogenic solution, wherein the aptamer HIS3-T2 has a nucleotide sequence shown in SEQ ID NO.2.

4. The aquatic product freshness enzyme sensor according to claim 3, wherein the aquatic product freshness enzyme sensor consists of the following components: 10 μL of an aptamer HIS3-T2 solution at a concentration of 1 μM, 10 μL of a chitopentaose solution at a concentration of 0.33 μM, 90 μL of a nanozyme AuNPs@FeP solution at a concentration of 1.98 nM, and 50 μL of the TMB chromogenic solution.

5. The aquatic product freshness enzyme sensor according to claim 4, wherein the TMB chromogenic solution is prepared from TMB, H₂O₂, and an acetic acid buffer, wherein in the chromogenic solution, the TMB has a concentration of 2 mM, the H₂O₂ has a volume concentration of 20%, and the acetic acid buffer has a concentration of 1 M and pH of 4.0.

6. The aquatic product freshness enzyme sensor according to claim 4, wherein the nanozyme AuNPs@FeP solution is prepared by the following method:
preparing gold nanoparticles (AuNPs), said preparing gold nanoparticles comprising:
adding 1 mL of a chloroauric acid solution at a concentration of 10 mg/mL to 95 mL of ultrapure water;
heating to boiling;
adding 4 mL of a trisodium citrate solution at a concentration of 10 mg/mL; and
continuing heating until the solution turns a stable wine-red color to obtain an AuNPs solution; and
preparing the nanozyme AuNPs@FeP solution, said preparing the nanozyme AuNPs@FeP solution comprising:
taking 496 μL of the AuNPs solution;
adding 2 μL of a phosphate buffer at a concentration of 1 mM and with pH of 7.1;
stirring at room temperature for 3 min;
then adding 2 μL of an FeCl₂ solution at a concentration of 50 μM; and
reacting for 15 min to obtain the nanozyme AuNPs@FeP solution, wherein AuNPs@FeP has a concentration of 1.98 nM.

7. An application of the aquatic product freshness enzyme sensor according to claim 4 in detecting histamine.

8. An application of the aquatic product freshness enzyme sensor according to claim 4 in rapidly evaluating freshness of an aquatic product.

9. A method for rapidly evaluating freshness of an aquatic product, comprising:
using microneedle patches to perform pressing extraction on the aquatic product to be detected for 2 min;
then transferring the moist microneedle patches to a centrifuge tube containing 995 μL of a PBS buffer at a concentration of 1 mM;
centrifuging at 4500 rpm for 10 s; and
filtering to obtain a sample to be detected; and detecting a concentration of histamine in the sample to be detected by using the enzyme sensor according to claim 4.

10. The method for rapidly evaluating freshness of an aquatic product according to claim 9, wherein the microneedle patches are prepared by the following method: taking 13 g of a polyvinyl alcohol prepolymer, dissolving same in 100 mL of ultrapure water at 90° C., cooling to 60° C., and adding 0.5 g of hyaluronic acid; and then, pouring a mixed solution into a mold for forming a cavity array of the microneedle patches, defoaming for 10 min under a condition of −1.0 Mpa, and drying to obtain the microneedle patches.


SEQUENCE LISTING (2 sequences)

SEQ ID NO: 1 - DNA, 80 bp, synthetic construct
  source 1..80: mol_type = other DNA
tgagcccaagccctggtatgaaagtcgcggtaaggagcgtgcttggattctcgttgggctggcaggtctactttgggatc

SEQ ID NO: 2 - DNA, 41 bp, synthetic construct
  source 1..41: mol_type = other DNA
tgagcccaagccctggtatgaatggattctcgttgggctgg